(19) United States
(12) Patent Application Publication
(10) Pub. No.: US 2004/0167092 A1
(43) Pub. Date: Aug. 26, 2004

(54) Medicaments

(76) Inventors: Charanjit Bountra, Harlow (GB); Timothy James Dale, Stevenage (GB); Christopher John Gardner, Stevenage (GB); Julian James Reeves, Harlow (GB); Micheal John Sheehan, Ware (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/469,792

(22) PCT Filed: Mar. 6, 2002
(86) PCT No.: PCT/GB02/01025

(30) Foreign Application Priority Data
Mar. 6, 2001 (GB) 0105469.1

(51) Int. Cl. A61K 31/7076; A61K 31/52

(52) U.S. Cl. 514/46; 514/263.2

(57) ABSTRACT

The present invention relates to the use of adenosine A1 agonists having an agonist action at adenosine A1 receptors in the treatment of emesis.

The present invention is concerned with medicaments, and more particularly medicaments for use in the treatment of emesis.

A variety of compounds which are agonists at the adenosine A1 receptor have been described in the art. These include compounds described in published patent applications WO99/24449, WO99/24450, WO99/24451, WO99/24452, WO98/01459, EP0322242, GB2226027, EP222330, WO98/08855, WO94/0707, WO99I67262, EP0322242, WO97/43300, WO98/16539 (Novo Nordisk A/S); WO98/04126 (Rhone-Poulenc Rorer Pharmaceuticals Inc.); WO98/01459 (Novo Nordisk A/S) and WO00/23447 (Aventis Pharmaceuticals). WO 99/67262 describes adenosine derivatives for the treatment of a patient suffering from a condition where there is an advantage in decreasing plasma free fatty acid concentration, or reducing heart rate or which subject is suffering from or susceptible to ischaemic heart disease, peripheral vascular disease or stroke or which subject is suffering pain, a CNS disorder or sleep apnoea.

U.S. Pat. No. 5,679,649 reported that adenosine phosphates give rapid recovery from anaesthesia without side effects such as nausea or vomiting. SU374086 reported that adenosine triphosphoric acid (ATP) could be used to treat air sickness. Other reports indicate that providing ATP reduces severity of radiation sickness. ATP may mediate this activity its own right or by being metabolised to adenosine diphosphate or adenosine monophosphate, all of which can act as agonists at cell surface purinoceptors of the P2 class, or by further metabolism to adenosine, which can act as an agonist at A1, A2a, A2b or A3 receptors. It is therefore unclear to the role if any played by adenosine receptors in emesis. Furthermore, other articles indicate that emesis is a side effect of adenosine agonists.

Surprisingly, the present inventors have found that compounds which are agonists at the adenosine A1 receptor, particularly partial agonists may be useful in the treatment of emesis.

There is also provided as a further aspect of the invention the use of an adenosine A1 agonist of the invention in the manufacture of a medicament for use in the treatment of emesis.

In an alternative or further aspect there is provided a method for the treatrnent of a mammal, including man, suffering from or susceptible to emesis, comprising administration of an effective amount of an adenosine A1 agonist of the invention.

It will be appreciated that reference to treatment is intended to include prophylaxis as well as the alleviation of established symptoms.

The treatment of emesis mentioned hereinbefore includes the treatment of nausea, retching and vomiting. Emesis includes acute emesis, delayed emesis and anticipatory emesis. Certain compounds having an agonist action at adenosine A1 receptors are useful in the treatment of emesis however induced.

For example, emesis may be induced by drugs such as cancer chemotherapeutic agents such as alkylating agents, e.g. cyclophosphamide, carmustine, lomustine and chlorambucil; cytotoxic antibiotics, e.g. dactinomycin, doxorubicin, mitomycin-C and bleomycin; anti-metabolites, e.g. cytarabine, methotrexate and 5-fluorouracil; vinca alkaloids, e.g. etoposide, vinblastine and vincristine; and others such as cisplatin, dacarbazine, procarbazine and hydroxyurea; and combinations thereof; radiation sickness; radiation therapy, e.g. irradiabon of the thorax or abdomen, such as in the treatment of cancer; poisons; toxins such as toxins caused by metabolic disorders or by infection, e.g. gastritis; pregnancy; vestibular disorders, such as motion sickness or vertigo; post-operative sickness; gastrointestinal obstruction; reduced gastrointestinal motility; visceral pain, e.g. myocardial infarction or peritonitis; migraine; increased intercranial pressure; decreased intercranial pressure (e.g. altitude sickness); and opioid analgesics, such as morphine.

The compounds useful in the invention are agonists at the adenosine A1 receptor. Preferably they are selective agonists at the adenosine A1 receptor. By selective is meant that the affinity for the A1 receptor is at least 2 times, preferably 5 times and more preferably 10 times greater than adenosine receptors A2a, A2b and A2c. Agonist selectivity of compounds against other human adenosine receptors was determined using Chinese hamster ovary (CHO) cells transfected with the gene for the relevant human adenosine receptor following a method based on that of Castanon, K V., and Spevak, W., (1994) Biochem. Biophys. Res. Commun. 198, 626-631. The CHO cells were also transfected with cyclic AMP response elements promoting the gene for secreted placental alkaline phosphatase (SPAP) (Wood, K V., (1995) Curr. Opinion. Biotechnology, 6, 50-58). The effect of test compounds was determined by their effects on basal levels of cAMP (A2a) or on forskolin-enhanced cAMP (Al and A3) as reflected by changes in levels of SPAP. EC₅₀ values for compounds were then determined as a ratio to that of the non-selective agonist N-ethyl carboxamidoadenosine (NECA).

The compounds may be partial agonists at the A1 receptor. A partial agonist is a compound that produced a maximal inhibitory response less than that for NECA in the spontaneously beating isolated rat right atrium preparation (preparation described in Gurden, M. F., Coates, J., Ellis, F., Evans, B., Foster, M., Homby, E., Kennedy, I., Martin, D P., Strong, P., Vardey, C J., et al. Functional characterisation of three adenosine receptor types. British Journal of Pharmacology 109(3): 693-8, 1993). More preferably partial agonist as a compound producing a max less than NECA in a FLIPR intracellular calcium release assay in CHO cells expressing a human adenosine A1 receptor. Sheehan, M. J., Wilson, D. J., Cousins, R. and Giles, H. (2000) Relative intrinsic efficacy of adenosine A1 receptor agonists measured using functional and radioligand binding assays. Brit. J. Pharmacol. 131, 34P.

Preferred compounds are for example described in WO99/24449, WO99/24450, WO99/24451, WO99/24452, WO98/01459, EP0322242, GB2226027, EP222330, WO98/08855, WO94/0707, WO99/67262, EP0322242, WO97/43300, WO98/16539 (Novo Nordisk A/S); WO98/04126 (Rhone-Poulenc Rorer Pharmaceuticals Inc.); WO98/01459 (Novo Nordisk A/S) and WO00/23447 (Aventis Pharmaceuticals), included herein by reference in their entirety.

Particularly preferred compounds of this invention are adenosine derivatives of formula (I) as disclosed in WO99/67262 (Glaxo Group Limited).

Other preferred compounds of the invention are N-(3-fluoro-4-hydroxyphenyl)-5′-O-methyl-adenosine and N-(4-hydroxyphenyl)-5′-O-methyl-adenosine.

The present invention therefore provides the use of an adenosine A1 agonist for the manufacture of a medicament for the treatment of emesis wherein the adenosine A1 agonist is selected from:

a compound of formula (I):

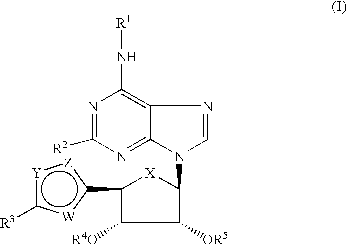

wherein X represents O or CH₂;

R² represents C₁₋₃alkyl, C₁₋₃alkoxy, halogen or hydrogen;

R³ represents H, phenyl (optionally substituted by halogen), a 5 or 6 membered heteroaryl group, C₁₋₆ alkoxy, C₁₋₆ alkylO(CH₂)ₙ where n is 0-6, C₃₋₇ cycloalkyl, C₁₋₆ hydroxyalkyl, halogen or a C₁₋₆ straight or branched alkyl, C₁₋₆ alkenyl or C₁₋₆ alkynyl group optionally substituted by one or more halogens;

Y and Z represent O, N, CH, N(C₁₋₆ alkyl);

W represents CH, O, N, S, N(C₁₋₆ alkyl);

and wherein at least one of W and Z represents a heteroatom (and when Y, Z and/or W is N, the presence or absence of an additional H would be apparent to a person skilled in the art);

with the proviso that when W represents CH, Z represents N and Y represents O, R³ cannot be H;

R⁴ and R⁵ independently represent H or a C₁₋₆ straight chain or branched alkyl group;

R¹ represents hydrogen or a group selected from:

(1) -(alk)ₙ—(C₃₋₇) cycloalkyl, including bridged cycloalkyl, said cycloalkyl group optionally substituted by one or more substituents selected from OH, halogen, —(C₁₋₃) alkoxy, wherein (alk) represents C₁₋₃ alkylene and n represents 0 or 1;

(2) an aliphatic heterocyclic group of 4 to 6 membered rings containing at least one heteroatom selected from O, N or S, optionally substituted by one or more substituents selected from the group consisting of —(C₁₋₃)alkyl, —CO2—C₁₋₄)alkyl, —CO(C₁₋₃alkyl), —S(═O)ₙ—(C₁₋₃alkyl), —CONR^{a}R^{b} (wherein R^{a} and R^{b} independently represent H or C₁₋₃alkyl) or ═O; where there is a sulfur atom in the heterocyclic ring, said sulfur is optionally substituted by (═O)ₙ, where n is 1 or 2;

(3) Straight or branched C₁₋₁₂ alkyl, optionally including one or more O, S(═O)ₙ (where n is 0, 1 or 2) and N groups substituted within the alkyl chain, said alkyl optionally substituted by one or more of the following groups, phenyl, halogen, hydroxy, C₃₋₇ cycloalkyl or NR^{a}R^{b} wherein R^{a} and R^{b} independently represent hydrogen, C₃₋₇ cycloalkyl or a C₁₋₆ straight chain or branched alkyl optionally substituted by C₃₋₇ cycloalkyl;

(4) a fused bicyclic aromatic ring:

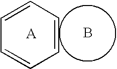

wherein B represents a 5 or 6 membered heterocyclic aromatic group containing 1 or more O, N or S atoms, wherein the bicyclic ring is attached to the nitrogen atom of formula (I) via a ring atom of ring A and ring B is optionally substituted by —CO₂—(C₁₋₃alkyl);

(5) a phenyl group optionally substituted by one or more subsfituents selected from:

-halogen, —SO₃H, -(alk)ₙOH, -(alk), -cyano, —(O), —(C₁₋₆)alkyl (optionally substituted by one or more halogens), - (alk)ₙ -nitro, —(O)ₘ -(alk)ₙ—CO₂R^{c},

-(alkₙ)- CONR^{c}R^{d} -(alk)ₙ —COR^{c}, -(alk)ⁿ —SOR^{e}, -(alk)ₙ —SO₂R^{e}, -(alk)ₙ- SO₂NR^{c}R^{d}, -(alk)ₙOR^{c}, -(alk)ₙ —(CO)ₘ- NHSO₂R^{e}, -(alk)ₙ- NHCOR_{c}, -(alk)ₙ- NR^{c}R^{d} wherein m and n are 0 or 1 and alk represents a C₁₋₆alkylene group or C₂₋₆ alkenyl group;

(6) A phenyl group substituted by a 5 or 6 membered heterocyclic aromatic group, said heterocyclic aromatic group optionally being substituted by C₁₋₃alkyl or NR^{c}R^{d};

R^{c} and R^{d} may each independently represent hydrogen, or C₁₋₃ alkyl or when part of a group NR^{c}R^{d}, R^{c} and R^{d} together with the nitrogen atom may form a 5 or 6 membered heterocyclic ring optionally containing other heteroatoms, which heterocyclic ring may optionally be substituted further by one or more C₁₋₃ alkyl groups;

R^{e} represents CI₃alkyl;

N-(3-fluoro4-hydroxyphenyl)-5′-O-methyl-adenosine; and

N-(4-hydroxyphenyl)-5′-O-methyl-adenosine;

or a salt and/or solvate thereof.

N-(3-fluoro4-hydroxy-phenyl)-5′-O-methyl-adenosine and N-(4-hydroxy-phenyl)-5′-O-methyl-adenosine are novel compounds and form a further aspect of this invention.

The present invention also provides a method of treatment of emesis in a mammal which comprises the administration of a therapeutically effective amount of compound of formula (I), N-(3-fluoro4-hydroxy-phenyl)-5′-O-methyl-adenosine, or N-(4-hydroxy-phenyl)-5′-O-methyl-adenosine to said mammal.

The present invention further provides for the use of an adenosine A1 agonist selected from a compound of formula (I), N-(3-fluoro-4-hydroxy-phenyl)-5′-O-methyl-adenosine and N-(4-hydroxy-phenyl)-5′-O-methyl-adenosine in the treatment of emesis.

It will be appreciated that wherein R¹ and/or R² in compounds of formula (I) contain one or more asymmetric carbon atoms the invention includes all diastereoisomers of compounds of formula (I) and mixtures thereof. Otherwise the stereochemical configuration of compounds of the invention is as depicted in formula (I) above.

As used herein, the term “alkyl” means a straight or branched chain alkyl group. Examples of suitable alkyl groups within R¹ and R² include methyl, ethyl, n-propyl, l-propyl, n-butyl, s-butyl, t-butyl and 2,2-dimethylpropyl.

As used herein, the term “alkylene” means a straight or branched chain alkylene group containing 1-6 carbon atoms, e.g. methylene.

As used herein, the term “C₂₋₆alkenyl” means a straight or branched chain alkenyl group containing 2 to 6 carbon atoms. Allyl represents an example of a suitable C₂₋₆alkenyl group.

The term “halogen” means fluorine, chlorine, bromine or iodine.

By aliphatic heterocyclic group defined for R¹ is meant a cyclic group of 4-6 carbon atoms wherein one or more of the carbon atoms is/are replaced by heteroatoms independently selected from nitrogen, oxygen or sulfur. This group may optionally be substituted as defined hereinabove.

The term heterocyclic aromatic group defined for R¹ refers to an aromatic mono or bicyclic ring system comprising from 5 to 10 carbon atoms wherein one or more of the carbon atoms is/are replaced by heteroatoms independently selected from nitrogen, oxygen and sulfur, which ring system may optionally be substituted as defined hereinabove.

Examples of W, Y and Z containing heterocyclic groups include isoxazoles, oxadiazoles, pyrazoles, oxazoles, triazoles and thiadiazoles.

Preferred W, Y and Z containing heterocyclic groups are isoxazoles, and 1,2,4- and 1,3;4- oxadiazoles.

R² preferably represents hydrogen, methyl, methoxy or halogen, more preferably hydrogen or chlorine.

Conveniently, R¹ may represent (alk)ₙ- C₃₋₆ cycloalkyl wherein n is 0 or 1 and the said cycloalkyl is either substituted by at least one substituent selected from halogen, particularly fluorine, and OH or is unsubstituted. Preferably n is zero. More preferably, the cycloalkyl group is unsubstituted or monosubstituted with OH and more preferably the cycloalkyl ring has 5 carbon members. Most preferably, the cycloalkyl group is hydroxycyclopentyl.

Alternatively R¹ may represent a substituted or unsubstituted aliphatic heterocyclic group, the substitutent being selected from the group consisting of —CO₂—(C₁₋₄)alkyl.

Conveniently, the aliphatic heterocyclic group is unsubstituted or when the substituent is —CO₂(C₁₋₄)alkyl, the heteroatom is N and the substituent is directly attached to said ring nitrogen atom.

Preferably the heterocyclic ring is 6 membered and more preferably contains only one 0, N or S heteroatom. Most preferably when the heterocyclic ring is unsubstituted the heteroatom is O. Most preferably when the heterocyclic ring is substituted the heteroatom is N.

Alternatively, R¹ may represent a straight or branched alkyl of 1-6 carbon atoms optionally with at least one S(═O)ₙ and where S(═O)ₙ is present, optionally substituted with N at a position adjacent to the S(═O)ₙ group; where there is an S(═O)ₙ in the chain, substitution with N at a position adjacent to the S(═O)ₙ group is preferred; where there is an S(═O)ₙ in the chain, preferably n is I or 2, more preferably n is 2. The alkyl group conveniently may be unsubstituted or substituted by at least one OH group.

Alternatively R¹ may represent a phenyl group which is substtuted by one or two substituents selected from OH, alkyl, particularly C₁₋₄ alkyl and halogen.

Preferably the phenyl is disubstituted in the 2,4 positions. Preferably both substituents are halogen more particularly, fluorine and chlorine. For example, a particularly preferred combination is 24luoro and 4-chloro. Preferably R⁴ and R⁵ represent hydrogen.

A particularly preferred compound of formula (I) is (2S,3S,4R,5R)-2-(5-tert-butyl-[1,3 ,4]-oxadiazol-2-yl)-5-[6-(4-chloro-2-fluorophenylamino)-purin-9-yl]tetrahydrofuran-3,4-diol, Example 14 of WO99/67262, the structure of which is indicated below:

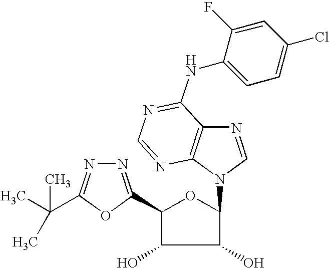

or pharmaceutically acceptable salts or solvates thereof.

Compounds of formula (I) are prepared according to processes disclosed in WO99/67262.

WO99/67262 is incorporated by reference herein as though fully set forth.

It will also be appreciated by those skilled in the art that the compounds of the present invention may also be utilized in the form of a pharmaceutically acceptable salt or solvate thereof. The physiologically acceptable salts of the compounds include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine and procaine salts. Those skilled in the art of organic chemistry will appreciate that many organic compounds can form complexes with solvents in which they are reacted or from which they are precipitated or crystallized. These complexes are known as “solvents”. For example, a complex with water is known as a “hydrate”. Solvates of the compounds are within the scope of the invention. References hereinafter to a compound according to the invention include both compounds and their pharmaceutically acceptable salts and solvates.

The compounds of the invention and their pharmaceutically acceptable derivatives are conveniently administered in the form of pharmaceutical compositions. Such compositions may conveniently be presented for use in conventional manner in admixture with one or more physiologically acceptable carriers or excipients.

While it is possible that compounds of the present invention may be therapeutically administered as the raw chemical, it is preferable to present the active ingredient as a pharmaceutical formulation. The carrier(s) must be “acceptable” in the sense of being compatible with the other ingredients of the formulation and not deleterious to the recipient thereof.

Accordingly, the present invention further provides for a pharmaceutical formulation comprising an adenosine A1 agonist in an amount effective for the treatment of emesis together with one or more pharmaceutically acceptable carriers therefore and, optionally, other therapeutic and/or prophylactic ingredients.

The formulations include those suitable for oral, parenteral (including subcutaneous e.g. by injection or by depot tablet, intradermal, intrathecal, intramuscular e.g. by depot and intravenous), rectal and topical (including dermal, buccal and sublingual) administration although the most suitable route may depend upon for example the condition and disorder of the recipient. The formulations may conveniently be presented in unit dosage fonm and may be prepared by any of the methods well known in the art of pharmacy. All methods include the step of bringing into association the compounds (“active ingredient”) with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets (e.g. chewable tablets in particular for paediatric administration) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a other conventonal excipients such as binding agents, (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate) or wetting agents, such as sodium lauryl sulfate. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The tablets may be coated according to methods well-known in the art.

Alternatively, the compounds of the present invention may be incorporated into oral liquid preparations such as aqueous or oily suspensions, solutions, emulsions, syrups or elixirs, for example. Moreover, formulations containing these compounds may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or acacia; non-aqueous vehicles (which may include edible oils) such as almond oil, fractionated coconut oil, oily esters, propylene glycol or ethyl alcohol; and preservatives such as methyl or propyl p-hydroxybenzoates or sorbic acid. Such preparations may also be formulated as suppositories, e.g., containing conventional suppository bases such as cocoa butter or other glycerides.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilised) condition requiring only the addition of a sterile liquid carrier, for example, water-for-injecbon, immediately prior to use. Extemporaneous injection solutions and suspensions may be prepared from sterile powders, granules and tablets of the kind previously described.

Formulations for rectal administration may be presented as a suppository with the usual carriers such as cocoa butter, hard fat or polyethylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

The compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In addition to the ingredients particularly mentioned above, the formulations may include other agents conventional in the art having regard to the type of formulation in question, for example those suitable for oral administration may include flavoring agents.

It will be appreciated by those skilled in the art that reference herein to treatment extends to prophylaxis (e.g. before stimuli known to predispose to emesis, e.g. travel sickness resulting from cancer chemotherapy as well as the treatment of established diseases or symptoms. Moreover, it will be appreciated that the amount of a compound of the invention required for use in treatment will vary with the nature of the condition being treated and the age and the condition of the patient and will be ultimately at the discretion of the attendant physician or veterinarian. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day. The formulations according to the invention may contain between 0.1-99% of the active ingredient, conveniently from 30-95% for tablets and capsules and 3-50% for liquid preparations.

The compound for use in the instant invention may be used in combination with other therapeutic agents for example other anti-emetic agents. The invention thus provides in a further aspect the use of a combination comprising an adenosine A1 agonist with a further therapeutic agent in the treatment of emesis.

When the compounds are used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a compound is used in combination with a second therapeutic agent active against the same disease, the dose of each compound may differ from that when the compound is used alone. Appropriate doses will be readily appreciated by those skilled in the art.

The present invention -will now be further illustrated by the accompanying examples which should not be construes as limiting the scope of the invention in any way.

### EXAMPLES

The effects of a number of A₁ agonists (including N-(3-fluoro4-hydroxy-phenyl)-5′-O-methyl-adenosine and N-(4-hydroxy-phenyl)-5′-O-methyl-adenosine) and an antagonist, ₁,3-dipropyl-8-cyclopentylxanthine (DPCPX), were investigated for anti-emetic action against radiaton-induced emesis in the conscious ferret. In addition DPCPX was used in an attempt to block the anti- emetic effects of the partial agonist, N-(3-fluoro-4hydroxy-phenyl)-5′-O-methyl-adenosine, the structure of which is indicated below.

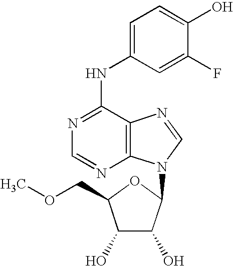

The anti-emetic effects of N-(3-fluoro-4hydroxy-phenyl)-5′-O-methyl-adenosine. were further investigated against morphine- and cisplatin-induced emesis in the conscious ferret. This partial adenosine A1 agonist N-(3-fluoro4-hydroxy-phenyl)-5′-O-methyl-adenosine may be prepared, for example by following methods described in WO97/43300 for example from the intermediate (2R,3R,4S,5R)-2-(6-chloro-purin-9-yl)-5-methoxymethyl-tetrahydro-furan-3,4-d iol described therein. A solution of this intermediate, 4-amino-2-fluorophenol (66mg) and diisopropylethylamine (0.15 mi) in isopropanol (12 mi) is stirred at reflux for 7 days then allowed to cool to room temperature. N-(4-hydroxy-phenyl)-5′-O-methyl-adenosine may be prepared using similar methodology.

### Method

For all the experiments. adult male ferrets, body weight range 1-1.5 kg, were used. Emesis was induced by X-irradiation, morphine and cisplatin.

X-irradiation:

The ferrets were weighed on the day before the experiment. On the day of the experiment each ferret received 2Gy (200 Rad) whole body X-irradiation, administered over a 5 minute period. The A1 receptor agonists, partial agonists or antagonist were administered via the subcutaneous route immediatelv after X-irradiation (i.e. approximately 25 minutes before the onset of emesis). When investigating the effects of the A1 receptor antagonist, DPCPX, on the partial agonist N-(3-fluoro4-hydroxy-phenyl)-5′-O-methyl-adenosine, either both compounds were administered simultaneously immediately after X-irradiation, or the DPCPX was given as a 15 minute pre-treatment. In all cases, the ferrets were observed for 2 hours after X-irradiation, and the time and numbers of retches and vomits were recorded.

Morphine:

The ferrets were weighed on the day before the experiment. The A₁ receptor partial agonist N-(3-fluoro4-hydroxy-phenyl)-5′-O-methyl-adenosine was administered subcutaneously 15 minutes before the 0.5 mg kg⁻¹ subcutaneous dose of morphine. (Emesis normally starts 5 minutes after morphine administration). The ferrets were observed for 2 hours after the morphine dose and the time and numbers of retches and vomits were recorded.

Cisplatin:

The ferrets were weighted and measured for the calculation of body surface area on the day before the experiment. On the day of the experiment each ferret received an intraperitoneal dose of cisplatin (200 mg m⁻²). The A₁ receptor partial agonist, N-(3-fluoro4-hydroxy-phenyl)-5′-O-methyl-adenosine, was administered subcutaneously immediately after the first emetic episode (approximately 1.5 hours after cisplatin administration). The ferrets were observed for 7 hours after the first emetic episode and the time and numbers of retches and vomits were recorded.

The effects of DPCPX, an A₁ receptor antagonist, on the anti-emetic
effect of N-(3-fluoro-4-hydroxy-phenyl)-5′-O-methyl-adenosine, an A₁
receptor partial agonist

| | Dose | % inhibition | |
|---|---|---|---|
| Compound | (mg kg-1) | of retching | n |
| [symbol] | [symbol] | [symbol] | [symbol] |
| N-(3-Fluoro-4-hydroxy- | 1.0 | 100 | 2 |
| phenyl)-5′-O-methyl- | | | |
| adenosine | | | |
| N-(3-Fluoro-4-hydroxy- | 1.0 | 68 | 2 |
| phenyl)-5′-O-methyl- | + | | |
| adenosine | 1.0 | | |
| + | | | |
| DPCPX | | | |
| (simultaneous) | | | |
| N-(3-Fluoro-4-hydroxy- | 1.0 | 67 | 2 |
| phenyl)-5′-O-methyl- | + | | |
| adenosine | 3.0 | | |
| + | | | |
| DPCPX | | | |
| (15 min pretreatment) | | | |

The effects of the A₁, receptor partial agonist, N-(3-Fluoro-4-hydroxy-
phenyl)-5′-O-methyl-adenosine, 1 mg kg⁻¹ s.c., on cisplatin- and
morphine-induced emesis

| | % inhibition of retching | | | |
|---|---|---|---|---|
| Emetogen | 1h | 2h | 7h | n |
| Cisplatin | 100% | 67% | 43% | 2 |
| Morphine | 100% | 100 | — | 2 |

### DISCUSSION

The full A₁ receptor agonists reduced radiation-induced emesis in the conscious ferret although not significantly. However, two of the seven partial agonists tested (N-(3-fluoro4-hydroxy-phenyl)-5′-O-methyl-adenosine and N-(4-hydroxy-phenyl)-5′-O-methyl-adenosine) completely inhibited emesis induced by [symbol] of one of the partial agonists, N-(3-fluoro4-hydroxy-phenyl)-5′-O-methyl-adenosine was shown to be partially reversed by the addition of the A₁ receptor antagonist, DPCPX. When a dose of 1 mgkg⁻¹ s.c. of DPCPX was administered simultaneously with N-(3-fluoro-4-hydroxy-phenyl)-5′-O-methyl-adenosine the 5 result was unconvincing as the anti-emetic effect in only one of the two animals tested was reduced. However, when the dose of DPCPX was increased to 3 mg kg⁻¹ s.c. and a 15 minute pre-treatment time was used the anti-emetic effect in both animals was reduced.

The results obtained provide evidence for the possible involvement of adenosine A₁ receptors in the emetic reflex. The anti-emetic effects of one of the partial agonists was attenuated by DPCPX, an antagonist, suggesting that inhibition of emesis resulted from agonist properties. The inhibition of emesis induced by the centrally-acting emetogen, morphine, suggests a central site of action for the A₁ receptor partial antagonists, and also a possible role for adenosine receptor partial agonists in the treatment of post-operative nausea and vomiting. Partial agonists were shown to be particularly effective ;in these studies possibly because they have a reduced cardiovascular side-effect liability compared to full agonists and can therefore be administered at doses high enough to more efficiently access the brain. Therefore full agonists would also be effective if they have physicochemical properties or are formulated in a manner which facilitates transport across the blood-brain barrier.

The application of which this description and claims forms part may be used as a basis for priority in respect of any subsequent application. The claims of such subsequent application may be directed to any novel feature or combination of features described herein. This may take the form of product, composition, process or use claims and may include, by way of example and without limitation, one or more of the following claims.

1. Use of an adenosine A1 agonist for the manufacture of a medicament for the treatment of emesis wherein the adenosine A1 agonist is selected from: a compound of formula (I): 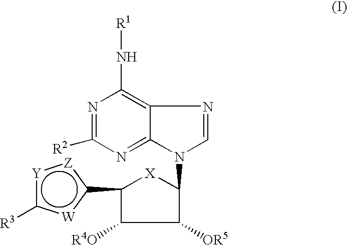
wherein X represents O or CH₂;
R² represents C_{1 3}alkyl, CI₃alkoxy, halogen or hydrogen;
R³ represents H, phenyl (optionally substituted by halogen), a 5 or 6 membered heteroaryl group, C₁₋₆ alkoxy, C₁₋₆ alkylO(CH₂)ₙ where n is 0-6, C₃₋₇cycloalkyl, C₁₋₆ hydroxyalkyl, halogen or a C₁₋₆ straight or branched alkyl, C₁₋₆ alkenyl or C₁₋₆ alkynyl group optionally substituted by one or more halogens;
Y and Z represent O, N, CH, N(C₁₋₆ alkyl);
W represents CH, O, N, S, N(C₁₋₆ alkyl);
and wherein at least one of W and Z represents a heteroatom (and when Y, Z and/or W is N, the presence or absence of an additional H would be apparent to a person skilled in the art);
with the proviso that when W represents CH, Z represents N and Y represents O, R³ cannot be H;
R⁴ and R⁵ independently represent H or a C₁₋₆ straight chain or branched alkyl group;
R¹ represents hydrogen or a group selected from:
(1) -(alk)ₙ -(C₃₋₇) cycloalkyl, including bridged cycloalkyl, said cycloalkyl group optionally substituted by one or more substituents selected from OH, halogen, -(C₁₋₃) alkoxy, wherein (alk) represents C₁₋₃ alkylene and n represents 0 or 1;
(2) an aliphatic heterocyclic group of 4 to 6 membered rings containing at least one heteroatom selected from O, N or S. optionally substituted by one or more substituents selected from the group consisting of -(C₁₋₃)alkyl, —CO₂—(C₁₋₄)alkyl, —CO(C₁₋₃alkyl), —S(═O)ₙ-(C₁₋₃alkyl), —CONR^{a}R^{b} (wherein R^{a} and R^{b} independently represent H or C₁₋₃alkyl) or ═O; where there is a sulfur atom in the heterocyclic ring, said sulfur is optionally substituted by (═O)ₙ, where n is 1 or 2;
(3) Straight or branched C₁₋₁₂ alkyl, optionally including one or more O, S(═O)ₙ (where n is 0, 1 or 2) and N groups substituted within the alkyl chain, said alkyl opffonally substituted by one or more of the following groups, phenyl, halogen, hydroxy, C₃₋₇ cycloalkyl or NR^{a}R^{b} wherein R^{a} and R^{b} independently represent hydrogen, C₃₋₇ cycloalkyl or a C₁₋₆ straight chain or branched alkyl optionally substituted by C₃₋₇ cycloalkyl;
(4) a fused bicyclic aromatic ring: 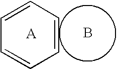
wherein B represents a 5 or 6 membered heterocyclic aromatic group containing 1 or more O, N or S atoms, wherein the bicyclic ring is attached to the nitrogen atom of formula (I) via a ring atom of ring A and ring B is optionally substituted by —CO₂ -(C₁₋₃alkyl);
(5) a phenyl group optionally substituted by one or more substituents selected from:
-halogen, —SO₃H, -(alk)ₙOH, -(alk)ₙ -cyano, —(O)ₙ -(C₁₋₆)alkyl (optionally substituted by one or more halogens), - (alk)ₙ -nitro, —(O)ₘ -(alk)ₙ—CO₂R^{c},
-(alkₙ)- CONR^{c}R^{d} -(alk)ₙ —COR^{c}, -(alk)ₙ —SOR^{e}, -(alk)ₙ —SO₂R^{e}, -(alk)ₙ- SO₂NR^{c}R^{d}, -(alk)ₙOR^{c}, -(alk), —(CO)^{m}- NHSO₂R^{e}, -(alk)ₘ- NHCOR^{c}, -(alk)ₙ- NR^{c}R^{d} wherein m and n are 0 or 1 and alk represents a C₁₋₆alkylene group or C₂₋₆ alkenyl group;
(6) A phenyl group substituted by a 5 or 6 membered heterocyclic aromatic group, said heterocyclic aromatic group optionally being substituted by C₁₋₃alkyl or NR^{c}R^{d};
R^{c} and R^{d} may each independently represent hydrogen, or C₁₋₃ alkyl or when part of a group NR^{c}R^{d}, R^{c} and R^{d} together with the nitrogen atom may form a 5 or 6 membered heterocyclic ring optionally containing other heteroatoms, which heterocyclic ring may optionally be substituted further by one or more C₁₋₃ alkyl groups; R^{e} represents C₁₋₃alkyl;
N-(3-fluoro-4-hydroxyphenyl)-5′-O-methyl-adenosine,iand
N-(4-hydroxyphenyl)-5′-O-methyl-adenosine;
or a salt and/or solvate thereof.

2. The use according to claim 1 wherein the adenosine A1 agonist is (2S,3S,4R,5R)-2-(5-tert-butyl-[1 ,3,4]-oxadiazol-2-yl)-5-[6-(4-chloro-2-fluorophenylamino)-purin-9-yl]-tetrahydrofuran-3,4-diol, or a salt and/or solvate thereof.

3. The use according to claim 1 or claim 2 wherein the medicament is formulated in unit doses:

4. A method of treatment of emesis in a mammal which comprises the administration of a therapeutically effective amount of compound of formula (I) according to claim 1, N-(3-fluoro4-hydroxy-phenyl)-5′-O-methyl-adenosine, or N-(4-hydroxy-phenyl)-5′-O-methyl-adenosine to said mammal.

5. Use of an adenosine A1 agonist selected from a compound of formula (I) according to claim 1, N-(3-fluora-4-hydroxy-phenyl)-5′-O-methyl-adenosine and N-(4-hydroxy-phenyl)-5′-O-methyl-adenosine in the treatment of emesis.

6. N-(3-fluoro4-hydroxy-phenyl)-5′-O-methyl-adenosine or N-(4-hydroxy-phenyl)-5′-O-methyl-adenosine.
